(19) United States
(12) Patent Application Publication
(10) Pub. No.: US 2021/0260015 A1
(43) Pub. Date: Aug. 26, 2021

(54) COMPOSITION FOR INHIBITING FAT ACCUMULATION

(71) Applicants: National University Corporation Tokyo University of Agriculture and Technology, Tokyo (JP); THE FOOD SCIENCE INSTITUTE FOUNDATION, Tokyo (JP); MEIJI CO., LTD., Tokyo (JP)

(72) Inventors: Ikuo KIMURA, Tokyo (JP); Daisuke KOZUTSUMI, Tokyo (JP); Kinya ASHIDA, Tokyo (JP); Kentaro NAKAMURA, Tokyo (JP)

(73) Assignees: National University Corporation Tokyo University of Agriculture and Technology, Tokyo (JP); THE FOOD SCIENCE INSTITUTE FOUNDATION, Tokyo (JP); MEIJI CO., LTD., Tokyo (JP)

(21) Appl. No.: 17/256,785

(22) PCT Filed: Jul. 2, 2019
(86) PCT No.: PCT/JP2019/026313

§ 371 Date: Dec. 29, 2020

(30) Foreign Application Priority Data
Jul. 3, 2018 (JP) 2018-126545

(51) Int. Cl. A61K 31/23; A61P 3/04; A23L 33/12; A23L 33/00; A23D 9/00

(52) U.S. Cl. CPC A61K 31/23; A61P 3/04; A23V 2002/00; A23L 33/40; A23D 9/00; A23L 33/12

(57) ABSTRACT

An object of the present invention is to provide a novel composition for use in suppressing fat accumulation, a novel composition for use in suppressing body weight gain, and a composition for use in promoting the production of a ketone body. According to the present invention, there is provided a composition for use in suppressing fat accumulation, suppressing body weight gain and/or promoting the production of a ketone body, comprising, as an active ingredient, a medium chain fatty acid glyceride comprising a saturated fatty acid having 8 carbon atoms as a constituent fatty acid. The ratio of the amount of the saturated fatty acid having 8 carbon atoms to the total amount of medium chain fatty acids of the medium chain fatty acid glyceride can be set to 0.6 or more. The medium chain fatty acid glyceride may further comprise, as a constituent fatty acid, a saturated fatty acid having 10 carbon atoms and/or a saturated fatty acid having 12 carbon atoms. The medium chain fatty acid glyceride is preferably a medium chain fatty acid triglyceride.

### CROSS-REFERENCE TO RELATED APPLICATION

The present application enjoys the benefit of priority from the prior Japanese Patent Application No. 2018-126545 filed on Jul. 3, 2018, the entire disclosure of which is incorporated herein by reference.

### TECHNICAL FIELD

The present invention relates to a composition for use in suppressing fat accumulation. The present invention also relates to a composition for use in suppressing body weight gain and a composition for use in promoting the production of a ketone body.

### BACKGROUND ART

In recent years, fatness due to overeating, lack of exercise and the like has become a social problem. Fatness refers to a condition in which fat is excessively accumulated in the body (a BMI of 25 or more), and is called obesity when a person develops a disease that adversely affects health due to fatness, or when a person develops visceral fat obesity. Fatness may cause diabetes, dyslipidemia (hyperlipemia), hyperuricemia (gout), cardiovascular and cerebrovascular diseases, fatty liver, menstruation disorder, knee pain, low back pain, sleep apnea syndrome, and the like. Since fatness causes various diseases, extensive research for prevention and improvement thereof has been made, and various treatment methods such as diet therapy, exercise therapy and drug therapy have been developed.

Of these therapies, in the diet therapy, food ingredients that are likely to be decomposed efficiently into energy have attracted attention, from the viewpoint of energy consumption. It has been reported that medium chain fatty acids, which are contained mainly in palm plants, are likely to be decomposed efficiently into energy when ingested in the body, as compared with long chain fatty acids which are the main components of common edible oils, and thus that body fat is less accumulated as compared with when long chain fatty acids are ingested (Non-Patent Document 1).

However, the relationship between the number of carbon atoms of a constituent fatty acid that binds to a medium chain fatty acid glyceride and the effect for suppressing fat accumulation has not been reported so far.

### REFERENCE LIST

### Non-Patent Document

Non-Patent Document 1: Bach A C et al., J. Lipid Res. 37(4): 708-726 (19%)

### SUMMARY OF THE INVENTION

An object of the present invention is to provide a novel composition for use in suppressing fat accumulation and a novel agent for suppressing fat accumulation. Another object of the present invention is to provide a novel composition for use in suppressing body weight gain and a novel agent for suppressing body weight gain. Still another object of the present invention is to provide a novel composition for use in promoting the production of a ketone body and a novel agent for promoting the production of a ketone body.

The present inventors have found that a medium chain fatty acid glyceride comprising a saturated fatty acid having 8 carbon atoms as a constituent fatty acid, when fed to mice, significantly suppresses fat accumulation and also significantly suppresses body weight gain. Also, the present inventors have found that the medium chain fatty acid glyceride, when fed to mice, significantly increases the plasma ketone body level of the mice. The present invention is based on these findings.

According to the present invention, the following inventions are provided.

[1] A composition for use in suppressing fat accumulation, for use in suppressing body weight gain and/or for use in promoting the production of a ketone body, and an agent for suppressing fat accumulation, an agent for suppressing body weight gain and/or an agent for promoting the production of a ketone body, each comprising, as an active ingredient, a medium chain fatty acid glyceride comprising a saturated fatty acid having 8 carbon atoms as a constituent fatty acid.

[2] The composition and agent according to [1], wherein the ratio (by mass, in terms of solid content) of the amount of the saturated fatty acid having 8 carbon atoms to the total amount of medium chain fatty acids of the medium chain fatty acid glyceride is 0.6 or more.

[3] The composition and agent according to [1] or[2], wherein the medium chain fatty acid glyceride further comprises, as a constituent fatty acid, a saturated fatty acid having 10 carbon atoms and/or a saturated fatty acid having 12 carbon atoms.

[4] The composition and agent according to any one of [1] to [3], which are each provided to a subject so that the medium chain fatty acid glyceride is ingested in a daily amount of 3 g or more (in terms of solid content).

[5] The composition and agent according to any one of [1] to [4], which are each provided to a subject so that the medium chain fatty acid glyceride is ingested in an amount equivalent to 1 to 95% kcal of the total lipid energy ingestion amount of the subject.

[6] The composition and agent according to any one of [1] to [5], which each further comprise a long chain fatty acid glyceride.

[7] The composition and agent according to [6], wherein the ratio (by mass, in terms of solid content) of the medium chain fatty acid glyceride to the long chain fatty acid glyceride in the composition is 1:0.1 to 1.5.

[8] The composition and agent according to any one of [1] to [7], which each further comprise a protein and/or a saccharide.

[9] The composition and agent according to [8], wherein the ratio (by mass, in terms of solid content) of the total mass of the medium chain fatty acid glyceride and the long chain fatty acid glyceride to the total mass of the protein and the saccharide is 0.1 or more.

[10] The composition and agent according to [8] or [9], wherein the contents of the medium chain fatty acid glyceride, the long chain fatty acid glyceride, the protein and the saccharide in the composition are 30 to 90% kcal, 5 to 50% kcal, 0 to 15% kcal and 0 to 10% kcal, respectively, with respect to the energy of the entire composition.

[11] The composition and agent according to any one of [1] to [10], wherein the medium chain fatty acid glyceride is a medium chain fatty acid triglyceride.

[12] The composition according to any one of [6] to [11], wherein the long chain fatty acid glyceride is a long chain fatty acid triglyceride.

[13] The composition and agent according to any one of [1] to [12], which are each a food composition.

[14] The composition and agent according to any one of [1] to [13], which are each a nutritional composition.

[15] The composition and agent according to any one of [1] to [13], which are each an oil and/or fat substitute.

[16] A composition for use in treating, preventing or improving obesity, and an agent for treating, preventing or improving obesity, each comprising, as an active ingredient, a medium chain fatty acid glyceride comprising a saturated fatty acid having 8 carbon atoms as a constituent fatty acid.

[17] A composition for use in reducing a risk of developing obesity and an agent for reducing a risk of developing obesity, each comprising, as an active ingredient, a medium chain fatty acid glyceride comprising a saturated fatty acid having 8 carbon atoms as a constituent fatty acid.

[18] A method for suppressing fat accumulation, a method for suppressing body weight gain and a method for promoting the production of a ketone body, each comprising feeding or administering an effective amount of a medium chain fatty acid glyceride comprising a saturated fatty acid having 8 carbon atoms as a constituent fatty acid or a composition comprising the medium chain fatty acid glyceride to a subject in need thereof.

[19] A method for treating, preventing or improving obesity and a method for reducing a risk of developing obesity, each comprising feeding or administering an effective amount of a medium chain fatty acid glyceride comprising a saturated fatty acid having 8 carbon atoms as a constituent fatty acid or a composition comprising the medium chain fatty acid glyceride to a subject in need thereof.

[20] Use of a medium chain fatty acid glyceride comprising a saturated fatty acid having 8 carbon atoms as a constituent fatty acid, for the manufacture of an agent for suppressing fat accumulation, for the manufacture of an agent for suppressing body weight gain, for the manufacture of an agent for promoting the production of a ketone body, as an agent for suppressing fat accumulation, as an agent for suppressing body weight gain, or as an agent for promoting the production of a ketone body.

[21] Use of a medium chain fatty acid glyceride comprising a saturated fatty acid having 8 carbon atoms as a constituent fatty acid, for the manufacture of an agent for treating, preventing or improving obesity, for the manufacture of an agent for reducing a risk of developing obesity, as an agent for treating, preventing or improving obesity, or as an agent for reducing a risk of developing obesity.

[22] A medium chain fatty acid glyceride comprising a saturated fatty acid having 8 carbon atoms as a constituent fatty acid, for use in suppressing fat accumulation, for use in suppressing body weight gain, or for use in promoting the production of a ketone body.

[23] A medium chain fatty acid glyceride comprising a saturated fatty acid having 8 carbon atoms as a constituent fatty acid, for use in treating, preventing or improving obesity, or for use in reducing a risk of developing obesity.

The compositions of [1], [15] and [16] are each sometimes referred to as “composition of the present invention” herein, and the agents of [1], [15] and [16] are each sometimes referred to as “agent of the present invention” herein.

The compositions and agents of the present invention are advantageous in that they can effectively suppress fat accumulation and body weight gain.

### BRIEF DESCRIPTION OF THE DRAWINGS

FIG. 1 is a graph showing body weight changes of groups which ingested test diets blended with MCTs as compared with groups which ingested a normal diet and the like. The body weight is expressed as average t standard deviation. ** denotes p<0.01, and * denotes p<0.05 vs. LCT (Dunnett's test).

FIG. 2 is a graph showing weights of adipose tissues (adipose tissues around the testis and the kidney, subcutaneous adipose tissue and brown adipose tissue) 5 weeks after the beginning of ingestion of the groups which ingested the test diets blended with MCTs as compared with the groups which ingested the normal diet and the like. The adipose tissue weight is expressed as average±standard deviation. ** denotes p<0.01, * denotes p<0.05, and a denotes p<0.1 vs. LCT (Dunnett's test).

FIG. 3 is a graph showing plasma levels of ketone bodies (acetoacetic acid, 3-hydroxybutyric acid and total ketone body) 5 weeks after the beginning of ingestion of the groups which ingested the test diets blended with MCTs as compared with the groups which ingested the normal diet and the like. The ketone body level is expressed as average f standard deviation. * denotes p<0.05, and a denotes p<0.1 vs. LCT (Dunnett's test).

### DETAILED DESCRIPTION OF THE INVENTION

In the present invention, the “medium chain fatty acid glyceride” is a compound having a structure in which medium chain fatty acid(s) are ester-bonded to glycerol. A compound in which one fatty acid binds to glycerol is referred to as medium chain fatty acid monoglyceride (monoacylglycerol); a compound in which two fatty acids bind to glycerol is referred to as medium chain fatty acid diglyceride (diacylglycerol); a compound in which three fatty acids bind to glycerol is referred to as medium chain fatty acid triglyceride (triacylglycerol). In the present invention, the meaning of the “medium chain fatty acid glyceride” includes medium chain fatty acid monoglyceride, medium chain fatty acid diglyceride and medium chain fatty acid triglyceride, and the “medium chain fatty acid glyceride” may be any of medium chain fatty acid monoglyceride, medium chain fatty acid diglyceride and medium chain fatty acid triglyceride, or a combination of part or all of them. In the present invention, the “medium chain fatty acid glyceride” is preferably a medium chain fatty acid triglyceride (MCT: Medium Chain Triglyceride, sometimes referred to as “MCT” herein). Hereinafter, the descriptions about the medium chain fatty acid glyceride can be read as the descriptions about the medium chain fatty acid triglyceride in a preferred aspect of the present invention.

In the present invention, the “medium chain fatty acid” which is a constituent fatty acid of the medium chain fatty acid glyceride refers to a saturated fatty acid having 6 to 12 carbon atoms, and the number of carbon atoms preferably ranges from 8 to 12, more preferably ranges from 8 to 10. Examples of the medium chain fatty acid include hexanoic acid (caproic acid), heptanoic acid (enanthic acid), octanoic acid (caprylic acid), nonanoic acid (pelargonic acid), decanoic acid (capric acid), and dodecanoic acid (lauric acid). Further, the three medium chain fatty acids which bind to one glycerol may be the same or different.

The composition and agent according to the present invention each comprise, as an active ingredient, a medium chain fatty acid glyceride comprising a saturated fatty acid having 8 carbon atoms as a constituent fatty acid (sometimes referred to as “medium chain fatty acid glyceride of the present invention” herein). The medium chain fatty acid glyceride of the present invention may be a medium chain fatty acid glyceride comprising, as all of the constituent fatty acids, saturated fatty acids having 8 carbon atoms, or a medium chain fatty acid glyceride comprising, as part of the constituent fatty acids, saturated fatty acids having 8 carbon atoms. When the medium chain fatty acid glyceride of the present invention is a medium chain fatty acid triglyceride, the medium chain fatty acid glyceride of the present invention may be a medium chain fatty acid triglyceride comprising, as all of the constituent fatty acids, saturated fatty acids having 8 carbon atoms (i.e., a triglyceride in which all of the three constituent fatty acids binding to glycerol are saturated fatty acids having 8 carbon atoms), or a medium chain fatty acid triglyceride comprising, as part of the constituent fatty acids, saturated fatty acids having 8 carbon atoms (i.e., a triglyceride in which one or two of the three constituent fatty acids binding to glycerol is/are a saturated fatty acid/saturated fatty acids having 8 carbon atoms). In this case, the constituent fatty acid other than the saturated fatty acid having 8 carbon atoms can be a saturated fatty acid having 6 or more and 12 or less carbon atoms, and may be preferably either or both of a saturated fatty acid having 10 carbon atoms and a saturated fatty acid having 12 carbon atoms.

The proportion of the saturated fatty acid having 8 carbon atoms contained in the medium chain fatty acid glyceride of the present invention can be expressed as the ratio (by mass, in terms of solid content) of the amount of the saturated fatty acid having 8 carbon atoms to the total amount of the medium chain fatty acids of the medium chain fatty acid glyceride. The lower limit value of this ratio can be set to 0.6 from the viewpoint of more satisfactorily exerting the effects of the present invention, and is preferably 0.7, more preferably 0.8, particularly preferably 0.9. The upper limit value of the ratio can be set to 1.0, and is preferably 0.98 or 0.95. The lower limit value and upper limit value can be arbitrarily combined, and the range of the ratio can be set to, for example, 0.6 to 1.0 or 0.7 to 0.98. The “solid content.” as used herein, means a component from which moisture has been removed.

The proportion of the saturated fatty acid having 10 carbon atoms and/or the saturated fatty acid having 12 carbon atoms contained in the medium chain fatty acid glyceride of the present invention can be expressed as the ratio (by mass, in terms of solid content) thereof to the saturated fatty acid having 8 carbon atoms. The ratio (by mass, in terms of solid content) of the saturated fatty acid having 8 carbon atoms to the saturated fatty acid having 10 carbon atoms in the medium chain fatty acid glyceride of the present invention can be set to for example 1:0.02 to 0.67, preferably 1:0.02 to 0.43 or 1:0.05 to 0.43. The ratio (by mass, in terms of solid content) of the saturated fatty acid having 8 carbon atoms to the saturated fatty acid having 12 carbon atoms in the medium chain fatty acid glyceride of the present invention can be set to for example 1:0.02 to 0.67, preferably 1:0.02 to 0.43, more preferably 1:0.05 to 0.43. The ratio (by mass, in terms of solid content) among the saturated fatty acid having 8 carbon atoms, the saturated fatty acid having 10 carbon atoms and the saturated fatty acid having 12 carbon atoms in the medium chain fatty acid glyceride of the present invention can be set to for example 1:0.3 to 50:0.3 to 50, preferably 1:0.5 to 30:0.5 to 30, further preferably 1:0.5 to 3:1 to 30.

Medium chain fatty acid glycerides are present in oils and/or fats contained in plant bodies such as palm plants including coconuts and palm fruits and dairy products such as milk. The medium chain fatty acid triglyceride of the present invention can employ a medium chain fatty acid glyceride extracted (including crude extraction) or purified (including rough purification) from these oils and/or fats (preferably, vegetable oils and/or fats such as palm kernel oil) as it is or as a raw material, as long as it is a medium chain fatty acid triglyceride comprising a saturated fatty acid having 8 carbon atoms as a constituent fatty acid. Alternatively, a product obtained by a chemical synthesis method or a commercially available product may be used as the medium chain fatty acid glyceride of the present invention. The medium chain fatty acid glyceride of the present invention may consist only of medium chain fatty acid glycerides having the same structure, or may be a mixture of medium chain fatty acid glycerides different in type of constituent fatty acid and/or binding position of constituent fatty acid to glycerol. From the viewpoint of stable manufacture and manufacturing cost, it is desirable to use a mixture of medium chain fatty acid glycerides different in type of constituent fatty acid and/or binding position of constituent fatty acid to glycerol.

The composition and agent according to the present invention can each comprise the medium chain fatty acid glyceride of the present invention alone, or can each comprise the medium chain fatty acid glyceride of the present invention in combination with any other component (for example, a food raw material, a food additive or a formulation additive). The content of the medium chain fatty acid glyceride of the present invention in the composition and agent according to the present invention can be arbitrarily determined depending, for example, on the purpose, intended use, form, dosage form, symptom, body weight and the like. In the present invention, the agent of the present invention can consist of the medium chain fatty acid glyceride of the present invention, and the composition of the present invention can comprise the medium chain fatty acid glyceride of the present invention.

The content of the medium chain fatty acid glyceride of the present invention in the composition and agent according to the present invention can be expressed as the ratio (e.g., by mass, energy ratio) of the medium chain fatty acid glyceride to the composition. The lower limit value of the content of the medium chain fatty acid glyceride of the present invention in the composition and agent according to the present invention can be set to, for example, 5% by mass, and is preferably 10% by mass, more preferably 20% by mass, further preferably 30% by mass. The upper limit value of the content can be set to 80% by mass, and is preferably 70% by mass, more preferably 60% by mass, further preferably 50% by mass. The lower limit value and upper limit value can be arbitrarily combined, and the range of the content can be set to, for example, 10 to 80% by mass (preferably 20 to 60% by mass, more preferably 30 to 50% by mass). The content of the medium chain fatty acid glyceride of the present invention in the composition and agent according to the present invention can also be expressed as the ratio with respect to the total energy amount of the composition and agent. The lower limit value of the ratio can be set to 30% kcal, and is preferably 35% kcal, more preferably 40% kcal. The upper limit value of the ratio can be set to 90% kcal, and is preferably 70% kcal, more preferably 55% kcal. The lower limit value and upper limit value can be arbitrarily combined, and the range of the ratio can be set to, for example, 30 to 90% kcal (preferably 40 to 70% kcal, more preferably 40 to 55% kcal).

The composition and agent according to the present invention may each further comprise a fatty acid glyceride (e.g., a long chain fatty acid glyceride) other than the medium chain fatty acid glyceride of the present invention, as will be described later. In this case, the lower limit value of the ratio (by mass, in terms of solid content) of the medium chain fatty acid glyceride of the present invention to all the fatty acid glycerides (oils and/or fats) in the composition and agent according to the present invention can be set to 0.4, and is preferably 0.5 or 0.55, and the upper limit value thereof can be set to 1.0, and is preferably 0.9 or 0.8. The lower limit value and upper limit value can be arbitrarily combined, and the range of the ratio (by mass, in terms of solid content) of the medium chain fatty acid glyceride of the present invention to all the fatty acid glycerides can be set to, for example, 0.4 to 1.0 or 0.5 to 0.9.

The composition and agent according to the present invention may each further comprise any of a long chain fatty acid glyceride, a protein and a saccharide, or a combination of part or all thereof. The composition and agent according to the present invention, which each further comprise a long chain fatty acid glyceride, a protein and a saccharide, can be provided as a food (for example, a nutritional food) imparted with the predetermined effects of the present invention. Hereinafter, the respective components will be described.

### (1) Long Chain Fatty Acid Glyceride

The composition and agent according to the present invention may each further comprise a long chain fatty acid glyceride as the fatty acid glyceride other than the medium chain fatty acid glyceride of the present invention. The “long chain fatty acid glyceride” is a compound having a structure in which a long chain fatty acid and glycerol are ester-bonded, and includes a long chain fatty acid monoglyceride in which one long chain fatty acid binds to glycerol, a long chain fatty acid diglyceride in which two long chain fatty acids bind to glycerol, and a long chain fatty acid triglyceride in which three long chain fatty acids bind to glycerol. The long chain fatty acid glyceride is preferably a long chain fatty acid triglyceride (LCT: Long Chain Triglyceride, sometimes referred to as “LCT” herein). Hereinafter, the descriptions about the long chain fatty acid glyceride can be read as the descriptions about the long chain fatty acid triglyceride in a preferred aspect of the present invention.

The “long chain fatty acid” which is a constituent fatty acid of the long chain fatty acid glyceride refers to a fatty acid having 13 to 30 carbon atoms, and the number of carbon atoms preferably ranges from 13 to 24, more preferably ranges from 13 to 18. Examples of long chain fatty acids include tetradecanoic acid, pentadecanoic acid, hexadecanoic acid, 9-hexadecenoic acid, octadecanoic acid, cis-9-octadecenoic acid, 11-octadecenoic acid, tetracosanoic acid, cis-15-tetracosanoic acid, and triacontanoic acid. The constituent fatty acid of the long chain fatty acid may be an unsaturated fatty acid. Further, the three long chain fatty acids which bind to one glycerol may be the same or different. When a plurality of types of long chain fatty acids bind to glycerol, their proportions are not particularly limited.

Long chain fatty acid glycerides are present in oils and/or fats contained in seeds of plant bodies such as soybean, rapeseed and olive, tallow, lard and the like. In an embodiment of the present invention, a long chain fatty acid glyceride extracted (including rough extraction) or purified (including rough purification) from such oils and/or fats can be used as the long chain fatty acid glyceride. Such oils and/or fats are commercially available, and commercially available products may be used.

The lower limit value of the content of the long chain fatty acid glyceride in the composition and agent according to the present invention can be set to, for example, 1% by mass, and is preferably 10% by mass, more preferably 25% by mass. The upper limit value of the content can be set to 45% by mass, and is preferably 42% by mass, more preferably 40% by mass. The lower limit value and upper limit value can be arbitrarily combined, and the range of the content can be set to, for example, 1 to 45% by mass (preferably 10 to 42% by mass, more preferably 25 to 40% by mass). The content of the long chain fatty acid glyceride in the composition and agent according to the present invention can also be expressed as the ratio with respect to the total energy amount of the composition and agent. The lower limit value of the ratio can be set to 5% kcal, and is preferably 20% kcal, more preferably 30% kcal. The upper limit value of the ratio can be set to 50% kcal, and is preferably 48% kcal, more preferably 45% kcal. The lower limit value and upper limit value can be arbitrarily combined, and the range of the ratio can be set to, for example, 5 to 50% kcal (preferably 20 to 48% kcal, more preferably 30 to 45% kcal).

The lower limit value of the ratio (by mass, in terms of solid content) of the long chain fatty acid glyceride to all the fatty acid glycerides in the composition and agent according to the present invention can be set to 0, but, from the viewpoint of blending an essential fatty acid, is preferably 0.1 or 0.2, and the upper limit value thereof can be set to 0.6, and is preferably 0.5 or 0.45. The lower limit value and upper limit value can be arbitrarily combined, and the range of the ratio (by mass, in terms of solid content) of the long chain fatty acid glyceride to all the fatty acid glycerides can be set to, for example, 0 to 0.6 or 0.1 to 0.5.

The ratio (by mass) of the medium chain fatty acid glyceride to the long chain fatty acid glyceride in the composition and agent according to the present invention can be set to 1:0.1 to 1.5 from the viewpoint of more satisfactorily exerting the effects of the present invention, and is preferably 1:0.25 to 1 or 1:0.25 to 0.8.

### (2) Protein

The composition and agent according to the present invention may each further comprise a protein. The protein used in the composition and agent according to the present invention is not particularly limited. Examples of the protein include corn gluten, wheat gluten, soybean protein, wheat protein, milk protein, animal protein (including collagen) obtained from meat or fish meat, egg white, and egg yolk, but milk protein is preferred. As the milk protein, protein components contained in milk obtained from mammals including primate animals such as humans, monkeys, gorillas, hamadryas baboons and chimpanzees; domestic animals such as horses, cows, buffaloes, sheep, goats, pigs, camels and deer; and pets such as dogs and cats can be used, and preferable protein components are preferably whey protein, casein and salts thereof.

Whey protein is a protein component contained in milk whey, and typical components include α-lactalbumin (α-La), β-lactoglobulin (β-Lg), immunoglobulin and lactoferrin. In the composition and agent according to the present invention, part or all of the above components can be used as the whey protein. Also, in the composition and agent according to the present invention, undiluted whey solutions (such as sweet whey and acid whey), concentrates thereof, dried products thereof (such as whey powder) and frozen products thereof can also be used as whey protein. Further, in the composition and agent according to the present invention, desalted whey, whey protein concentrates (WPCs) and whey protein isolates (WPIs) can also be used as the whey protein.

Casein is a kind of protein contained in milk in a proportion of about 80% by mass. When an acid is added to milk, precipitation occurs, and the resultant precipitate is casein. A casein salt is not particularly limited as long as it is allowed to be added to foods, and examples thereof include casein sodium, casein potassium, casein calcium and casein magnesium. Among these casein salts, casein sodium is preferred. Casein sodium can be manufactured, for example, by reacting casein with sodium hydroxide or sodium bicarbonate. Casein or casein salts is/are commercially available, and commercially available products may be used.

The lower limit value of the content of the protein in the composition and agent according to the present invention can be set to 0% by mass, and is preferably 5% by mass, more preferably 10% by mass. The upper limit value of the content can be set to 30% by mass, and is preferably 25% by mass, more preferably 20% by mass. The lower limit value and upper limit value can be arbitrarily combined, and the range of the content can be set to, for example, 0 to 30% by mass (preferably 5 to 25% by mass, more preferably 10 to 20% by mass). The content of the protein in the composition and agent according to the present invention can also be expressed as the ratio with respect to the total energy amount of the composition and agent. The lower limit value of the ratio can be set to 0% kcal, and is preferably 3% kcal, more preferably 5% kcal. The upper limit value of the ratio can be set to 15% kcal, and is preferably 12% kcal, more preferably 10% kcal. The lower limit value and upper limit value can be arbitrarily combined, and the range of the ratio can be set to, for example, 0 to 15% kcal (preferably 3 to 12% kcal, more preferably 5 to 10% kcal).

The daily ingestion amount (in terms of solid content) for a human (body weight: 50 kg) of the protein to be ingested together with the medium chain fatty acid glyceride of the present invention can be set to, for example, 1.0 g or more (preferably 1.5 g or more, more preferably 1.7 g or more), and the upper limit value thereof is not particularly limited, but, for example, can be set to 50 g or 30 g. The ingestion amount of the protein to be ingested together with the medium chain fatty acid glyceride of the present invention can also be set to be an amount equivalent to 0.1% kcal or more (preferably 0.15% kcal or more, more preferably 0.2% kcal or more) of the total energy ingestion amount (for example, per meal, per day or per week) of the subject, and the upper limit value thereof is not particularly limited, but, for example, can be set to be an amount equivalent to 50% kcal or 30% kcal. The content of the protein in the composition and agent according to the present invention may be determined based on this ingestion amount.

### (3) Saccharide

The composition and agent according to the present invention may each further comprise a saccharide. Saccharides are carbohydrates that are not dietary fibers, and include monosaccharides, disaccharides, oligosaccharides and polysaccharides. Examples of monosaccharides include glucose (grape sugar), fructose (fruit sugar), and galactose. Examples of disaccharides include maltose (malt sugar), sucrose (cane sugar), and lactose (milk sugar). Examples of oligosaccharides include galactooligosaccharide, fructooligosaccharide, and mannan oligosaccharide. Examples of polysaccharides include starch (amylose and amylopectin), glycogen, and dextrin. It should be noted that the saccharide preferably comprises lactose, more preferably comprises lactose but does not comprise glucose, further preferably substantially consists only of lactose.

The lower limit value of the content of the saccharide in the composition and agent according to the present invention can be set to 0% by mass, and is preferably 3% by mass, more preferably 5% by mass. The upper limit value of the content can be set to 15% by mass, and is preferably 13% by mass, more preferably 10% by mass. The lower limit value and upper limit value can be arbitrarily combined, and the range of the content can be set to, for example, 0 to 15% by mass (preferably 3 to 13% by mass, more preferably 5 to 10% by mass). The content of the saccharide in the composition and agent according to the present invention can also be expressed as the ratio with respect to the total energy amount of the composition and agent. The lower limit value of the ratio can be set to 0% kcal, and is preferably 2% kcal, more preferably 4% kcal. The upper limit value of the ratio can be set to 10% kcal, and is preferably 8% kcal, more preferably 6% kcal. The lower limit value and upper limit value can be arbitrarily combined, and the range of the ratio can be set to, for example, 0 to 10% kcal (preferably 2 to 8% kcal, more preferably 4 to 6% kcal).

The daily ingestion amount (in terms of solid content) for a human (body weight: 50 kg) of the saccharide to be ingested together with the medium chain fatty acid glyceride of the present invention can be set to, for example, 0.6 g or more (preferably 0.8 g or more, more preferably 0.95 g or more), and the upper limit value thereof is not particularly limited, but, for example, can be set to 30 g or 20 g. The ingestion amount of the saccharide to be ingested together with the medium chain fatty acid glyceride of the present invention can also be set to be an amount equivalent to 0.1% kcal or more (preferably 0.11% kcal or more, more preferably 0.12% kcal or more) of the total energy ingestion amount (for example, per meal, per day or per week) of the subject, and the upper limit value thereof is not particularly limited, but, for example, can be set to be an amount equivalent to 20% kcal or 10% kcal. The content of the saccharide in the composition and agent according to the present invention may be determined based on this ingestion amount.

The ratio of the total mass of the medium chain fatty acid glyceride and the long chain fatty acid glyceride to the total mass of the protein and the saccharide in the composition and agent according to the present invention (sometimes referred to as “ketone ratio” herein) can be arbitrarily determined as long as the effects of the present invention are provided. The lower limit value of the ketone ratio in the composition and agent according to the present invention can be set to 0.1 from the viewpoint of more satisfactorily exerting the effects of the present invention, and is preferably 1.0, more preferably 2.0, further preferably 2.5. The upper limit value of the ratio is not particularly limited, but can be set to 6.0, and is preferably 5.5, more preferably 5.0, further preferably 3.5. The lower limit value and upper limit value can be arbitrarily combined, and the range of the ratio can be set to, for example, 0.1 to 6, 2 to 5.5, or 2.5 to 3.5.

The compositional ratio among the fatty acid glycerides, the protein and the saccharide in the composition and agent (particularly, nutritional composition) according to the present invention can be defined as follows, from the viewpoint of more satisfactorily exerting the effects of the present invention. It should be noted that the compositional ratio is a ratio (percentage) with respect to the entire composition and agent, and the mass ratio is in terms of solid content.

### <Mass Ratio>

Medium chain fatty acid glyceride: 10 to 80% by mass (preferably, 20 to 60% by mass)
Long chain fatty acid glyceride: 1 to 45% by mass (preferably, 10 to 42% by mass)
Protein: 0 to 30% by mass (preferably, 5 to 25% by mass)
Saccharide: 0 to 15% by mass (preferably, 3 to 13% by mass)

### <Energy Ratio>

Medium chain fatty acid glyceride: 30 to 90% kcal (preferably, 40 to 70% kcal)
Long chain fatty acid glyceride: 5 to 50% kcal (preferably, 20 to 48% kcal)
Protein: 0 to 15% kcal (preferably, 3 to 12% kcal)
Saccharide: 0 to 10% kcal (preferably, 2 to 8% kcal)

The composition and agent according to the present invention are each used to suppress fat accumulation. The phrase “suppress fat accumulation” means suppressing accumulation of fat in the living body, and typically means suppressing accumulation of neutral fat in white adipose tissue of the living body. Examples of the accumulation of fat in the living body include accumulation of body fat and accumulation of visceral fat. Examples of the accumulation of body fat include accumulation of fat in subcutaneous adipose tissue, and examples of the accumulation of visceral fat includes accumulation of fat around organs (for example, kidney, testis, and mesentery). The degree of suppression of fat accumulation can be evaluated using, for example, the mass of white adipose tissue, the mass of white adipose tissue around an organ, the body fat percentage, or the BMI as an index.

The composition and agent according to the present invention are also each used to suppress body weight gain. The meaning of the phrase “suppress body weight gain” includes not only literally suppressing body weight gain but also maintaining or losing the body weight. In addition, the suppression of body weight gain also includes slimming. The degree of suppression of body weight gain can be evaluated using the body weight or the BMI as an index.

The composition and agent according to the present invention are also each used to promote the production of a ketone body. The phrase “promote the production of a ketone body” means increasing the level of a ketone body in the circulating blood of the subject, and the meaning thereof includes enhancing the production of a ketone body. Examples of the ketone body include acetoacetic acid and 3-hydroxybutyric acid. The degree of promotion of the production of a ketone body can be evaluated using the level(s) of one or more types of ketone bodies in the blood of the subject as an index. The level of a ketone body in the blood can be measured according to an enzyme method, a chromatograph method, or a method using a β-hydroxybutyric acid measuring device for self-examination (Precision Xceed, manufactured by Abbott).

The medium chain fatty acid glyceride of the present invention can suppress not only fat accumulation and but also body weight gain in a subject. Therefore, the composition and agent according to the present invention can be fed or administered to a subject suffering from obesity or a subject at a risk of suffering from obesity, thereby making it possible to treat, prevent or improve obesity. Therefore, the composition and agent according to the present invention can also be fed or administered to a subject at a risk of developing obesity, thereby making it possible to reduce the risk of developing obesity. The “subject at a risk of developing obesity” means a subject having no subjective symptoms of obesity but having a risk of developing obesity in the future due to factors such as eating habits, physical conditions, physical constitution, heredity, and exercise habits. In addition, the phrase “reduce a risk of developing obesity” means that the probability of developing obesity is reduced. Whether or not the subject suffers from obesity can be determined according to, for example, Guidelines for the Management of Obesity Disease 2016 (edited by the Japan Society for the Study of Obesity).

The composition and agent according to the present invention can be each provided in the form of a pharmaceutical product (for example, pharmaceutical composition), a quasi-drug, a food (for example, a food composition or a nutritional composition), a feed (for example, a pet food) or the like, and can be implemented according to the following descriptions.

The medium chain fatty acid glyceride of the present invention can be administered orally to a human and a non-human animal. Examples of oral drugs include granules, powders, tablets (including sugar-coated tablets), pills, capsules, syrups, emulsions, and suspensions. These formulations can be formulated using pharmaceutically acceptable carriers by techniques commonly used in the art. Pharmaceutically acceptable carriers include excipients, binders, diluents, additives, flavoring agents, buffers, thickeners, colorants, stabilizers, emulsifiers, dispersants, suspending agents, and preservatives.

In the present invention, administration of the medium chain fatty acid glyceride of the present invention to a human and a non-human animal other than oral administration (for example, tube administration or nasal tube administration) can also be performed according to the forms of the composition and agent according to the present invention. For example, the composition and agent according to the present invention are each prepared as a viscous liquid composition comprising the medium chain fatty acid glyceride of the present invention or a semi-solid composition comprising the medium chain fatty acid glyceride of the present invention, and thus can be fed or administered to a human and a non-human animal having impaired chewing and swallowing functions so that they cannot undergo oral feeding or oral administration. Even if the chewing and swallowing functions of the subject are impaired due to aging or the like, the effects for suppressing fat accumulation and the like can be expected in the subject by feeding or administering the composition and agent according to the present invention in a manner other than oral feeding.

The medium chain fatty acid glyceride of the present invention can be fed orally to a human and a non-human animal, in the present invention. When the medium chain fatty acid glyceride of the present invention is orally fed, it may be in an isolated, purified or roughly purified form, or in the form of a food or food raw material comprising the medium chain fatty acid glyceride of the present invention.

When the medium chain fatty acid glyceride of the present invention is provided as a food, it can be provided as it is or in a state of being blended in a food. The food thus provided is a food containing an effective amount of the medium chain fatty acid glyceride of the present invention. The phrase “containing an effective amount” of the medium chain fatty acid glyceride of the present invention refers to a content of the medium chain fatty acid glyceride of the present invention to be ingested within a range as will be described later, when a normally-eaten amount of individual foods is ingested. The meaning of the “food” includes health foods, functional foods, nutritional supplements, foods with health claims (such as foods for specified health uses, foods with nutrient function claims, and foods labeled with functions), foods for special dietary uses (such as foods for infants, foods for expectant and nursing mothers and foods for sick persons) and nutritional supplements (such as supplements). When the medium chain fatty acid glyceride of the present invention is fed to an animal other than a human, needless to say, the food referred to herein is used as a feed.

The form of the “food” is not particularly limited, and the food may be provided, for example, in a liquid form such as a beverage or a liquid diet, in a paste, semi-liquid or gelled form, or in a solid, bar or powder form. When the composition and agent according to the present invention are each used in a powder form, they can be manufactured by using a means such as spray drying or freeze drying.

The composition and agent according to the present invention can be each provided as a food containing the medium chain fatty acid glyceride of the present invention. When the food to be provided is a food containing an oil and/or fat as a raw material, the medium chain fatty acid glyceride of the present invention may be blended in addition to the raw material oil and/or fat, or blended in place of part or all of the raw material oil and/or fat. The latter food is advantageous in that the amount of oil and/or fat which can cause fat accumulation, body weight gain, obesity, and the like is reduced, that the medium chain fatty acid glyceride of the present invention, which has the effects for suppressing fat accumulation and the like, is blended, and thus that the food is provided as a food more potently imparted with at least any one of the effects for suppressing body weight gain, promoting the production of a ketone body, treating obesity and the like, and reducing a risk of developing obesity. In addition, the medium chain fatty acid glyceride of the present invention exhibits potent effects for suppressing fat accumulation and the like, and thus makes it possible to manufacture a food impaired with desired effects even if added in a small amount. Therefore, the medium chain fatty acid glyceride of the present invention is advantageous also from the viewpoints of flavor and manufacturing cost of the food.

When the composition and agent according to the present invention are each provided as a food containing the medium chain fatty acid glyceride of the present invention, they can be manufactured according to a normal food manufacturing method except that the medium chain fatty acid glyceride of the present invention is blended therein. Specifically, the food of the present invention can be prepared by adding the medium chain fatty acid glyceride of the present invention to various foods (for example, oils and/or fats, edible oils, powdered oils and/or fats, seasonings, margarine, butter, mayonnaise, cow's milk, refreshing drinks, fermented milk, yogurt, chocolate, gummy, cheese, bread, biscuits, cookies, crackers, pizza crusts, jellies, ice creams, soups, high-energy supplements, high-energy pastes, nutritional powders, powdered liquid diets, formulated milk powder, liquid diets, foods for special dietary uses, foods for sick persons, comprehensive nutritional foods, nutritional supplements, frozen foods, processed foods and other commercially available foods) or their raw materials, regardless of the form such as liquid, solid or powder.

Also, in the present invention, an effective amount of the medium chain fatty acid glyceride of the present invention is blended in various nutritional foods such as a ketogenic diet, and thus these foods can be provided as nutritional foods having both a fat accumulation suppressing function and the like and an energy supplementing function. In this case, the medium chain fatty acid glyceride of the present invention may be blended in addition to the raw material oil and/or fat of the nutritional foods or blended in place of part or all of the raw material oil and/or fat. The nutritional foods of the present invention can be provided in the form of liquid diets, and such foods are advantageous because they can be fed or administered to sick or elderly persons with impaired chewing and swallowing functions.

When the composition and agent according to the present invention are each provided as a supplement, they can be manufactured according to a normal supplement manufacturing method except that the medium chain fatty acid glyceride of the present invention is blended therein. Examples of the supplement include pastes obtained by adding an excipient, a thickener and the like to the medium chain fatty acid glyceride of the present invention and kneading them together, tablets manufactured by adding an excipient, a binder and the like to the medium chain fatty acid glyceride of the present invention, kneading them together and then tableting the kneaded product, and capsule agents in which the medium chain fatty acid glyceride of the present invention is encapsulated in a capsule and the like. Also, in the present invention, the subject himself/herself can add the medium chain fatty acid glyceride of the present invention having various properties (for example, liquid, solid, powder and paste forms) to water, a food or beverage, a meal or the like for ingestion thereof.

The composition and agent according to the present invention can also be each provided in the form of a food raw material (in particular, processed food raw material). When the food raw material to be provided is an oil and/or fat, the medium chain fatty acid glyceride of the present invention may be blended in addition to the oil and/or fat, or blended in place of part or all of the oil and/or fat. Examples of such food raw materials include edible oils and/or fats including vegetable oils such as corn oil, olive oil, almond oil, sesame oil, palm oil, coconut oil and cocoa butter; and animal oils such as butter, lard and tallow. Alternatively, the medium chain fatty acid glyceride of the present invention, itself, may be provided as the food raw material. Since the medium chain fatty acid glyceride of the present invention can be blended in foods as an oil and/or fat substitute, as described above, the composition and agent according to the present invention can be each provided as an oil and/or fat substitute or an additive for the oil and/or fat substitute.

The ingestion amount of the medium chain fatty acid glyceride of the present invention is not particularly limited because it varies depending on the age, body weight, symptoms, intended use and the like of the subject. When the medium chain fatty acid glyceride of the present invention is ingested for the purpose of suppressing fat accumulation, suppressing body weight gain, promoting the production of a ketone body, treating obesity and the like, and reducing a risk of developing obesity, the daily ingestion amount (in terms of solid content) for a human (body weight: 50 kg) of the medium chain fatty acid glyceride of the present invention can be set to, for example, 3 g or more (preferably 4 g or more, more preferably 4.5 g or more), and the upper limit value thereof is not particularly limited, but, for example, can be set to 65 g or 50 g. In this case, the subject preferably ingests the medium chain fatty acid glyceride of the present invention in place of part or all of the oil and/or fat other than the medium chain fatty acid glyceride of the present invention to be ingested by the subject. For example, when the daily ingestion amount (in terms of solid content) for a human of the medium chain fatty acid glyceride of the present invention is defined as A g, the daily ingestion amount of the oil and/or fat other than the medium chain fatty acid glyceride of the present invention to be ingested by the subject is preferably reduced by 0.8×A to 1.2×A g (preferably 0.9×A to 1.1×A g, more preferably about A g). In the present invention, examples of the oil and/or fat other than the medium chain fatty acid glyceride of the present invention to be ingested by the subject include edible oils and/or fats called visible oils (for example, edible oils, margarine, butter and mayonnaise) and oil and/or fat components contained in foods, called invisible oils (for example, animal oils and/or fats derived from meat, eggs, milk and fish, and vegetable oils and/or fats derived from grains, beans and confectioneries).

When the medium chain fatty acid glyceride of the present invention is ingested for the purpose of suppressing fat accumulation, suppressing body weight gain, promoting the production of a ketone body, treating obesity and the like, and reducing a risk of developing obesity, the medium chain fatty acid glyceride of the present invention can be ingested in an amount equivalent to 1% kcal or more (preferably 1.2% kcal or more, more preferably 1.5% kcal or more) of the total energy ingestion amount (for example, per meal, per day or per week) of the subject, and the upper limit value thereof is not particularly limited, but can be set to be an amount equivalent to 100% kcal or 90% kcal per meal, and can be set to an amount equivalent to 80% kcal or 70% kcal per day or per week. In this case, the subject preferably ingests the medium chain fatty acid glyceride of the present invention in place of part or all of the oil and/or fat other than the medium chain fatty acid glyceride of the present invention to be ingested by the subject. For example, when the daily ingestion amount of the medium chain fatty acid glyceride of the present invention is defined as an amount equivalent to B % kcal of the total daily energy ingestion amount for a human, the daily ingestion amount of the oil and/or fat other than the medium chain fatty acid glyceride of the present invention to be ingested by the subject is preferably reduced by an amount equivalent to 0.8×B to 1.2×B % kcal (preferably an amount equivalent to 0.9×B to 1.1×B % kcal, more preferably an amount equivalent to B % kcal) of the total daily energy ingestion amount for a human.

When the medium chain fatty acid glyceride of the present invention is ingested for the purpose of suppressing fat accumulation, suppressing body weight gain, promoting the production of a ketone body, treating obesity and the like, and reducing a risk of developing obesity, the medium chain fatty acid glyceride of the present invention can also be ingested in an amount equivalent to 1 to 95% kcal (preferably 2 to 80% kcal or more, more preferably 4 to 60% kcal or more) of the total lipid energy ingestion amount (for example, per meal, per day or per week) of the subject. In this case, the subject preferably ingests the medium chain fatty acid glyceride of the present invention in place of part or all of the oil and/or fat other than the medium chain fatty acid glyceride of the present invention to be ingested by the subject. For example, when the daily ingestion amount for a human of the medium chain fatty acid glyceride of the present invention is defined as an amount equivalent to C % kcal of the total daily lipid energy ingestion amount for a human, the daily ingestion amount of the oil and/or fat other than the medium chain fatty acid glyceride of the present invention to be ingested by the subject is preferably reduced by an amount equivalent to 0.8×C to 1.2×C % kcal of the total daily lipid energy ingestion amount for a human (preferably an amount equivalent to 0.9×C to 1.1×C % kcal, more preferably an amount equivalent to C % kcal).

All of the ingestion amounts described above may be appropriately increased or decreased depending on the contents of meals of the subject. The ingestion period can be set to usually 1 day or longer, preferably 3 days or longer, more preferably 1 week or longer. The number of times of ingestion is not particularly limited, and the effective ingestion amount may be ingested once daily or ingested in several batches. Also, the ingestion timing is not particularly limited, and the subject can ingest the medium chain fatty acid glyceride at a timing when it is easy for the subject to ingest it. The ingestion amount and ingestion timing of the medium chain fatty acid glyceride of the present invention described above and the ingestion period which will be described later are applicable when the medium chain fatty acid glyceride of the present invention is used for both non-therapeutic and therapeutic purposes, and the “ingestion” can be read as “administration” in the case of therapeutic purposes.

The medium chain fatty acid glyceride of the present invention can be fed to the subject together with a long chain fatty acid glyceride, in the present invention. The daily ingestion amount (in terms of solid content) for a human (body weight: 50 kg) of the fatty acid glycerides including the medium chain fatty acid glyceride of the present invention can be set to, for example, 6 g or more (preferably 8 g or more, more preferably 10 g or more). The ingestion amount of the fatty acid glycerides including the medium chain fatty acid glyceride of the present invention can be set to an amount equivalent to 1.3 to 92% kcal (preferably 1.5 to 90% kcal, more preferably 2 to 88% kcal) of the total energy ingestion amount (for example, per meal, per day or per week) of the subject.

The medium chain fatty acid glyceride of the present invention may be fed to the subject together with any of a long chain fatty acid glyceride, a protein and a saccharide, or a combination of part or all thereof. The ingestion amounts of the fatty acid glycerides including the medium chain fatty acid glyceride of the present invention, the protein and the saccharide are as follows, and the composition and agent according to the present invention may each comprise these components so that the components are ingested in the following amounts.

The composition and agent according to the present invention are each used in combination with any other orally ingestible composition and/or agent, with no limitation. For example, the composition and agent according to the present invention can be each used in combination with a material or composition that can be expected to suppress fat accumulation, suppress body weight gain, or promote the production of a ketone body to further enhance the effects of the present invention. The composition and agent according to the present invention each utilize, as an active ingredient, a medium chain fatty acid glyceride which is a food material, and thus have no fear of causing side effects even when used continuously. Therefore, the combination of the composition and agent according to the present invention with an existing agent for suppressing fat accumulation can reduce the dose of the existing agent and therefore can alleviate or overcome the side effects of the existing agent. When used in combination with any other agent, the composition and agent according to the present invention may be prepared separately from the other agent, or the other agent and the composition and agent according to the present invention (or the medium chain fatty acid glyceride of the present invention) may be blended together in the same composition.

The composition and agent according to the present invention can be each provided to give a daily ingestion amount effective for suppressing fat accumulation, suppressing body weight gain and promoting the production of a ketone body. In this case, the composition and agent according to the present invention may be each packaged so that the medium chain fatty acid glyceride of the present invention can be ingested in an effective daily ingestion amount. The package form may be a single package or a multiple package as long as the effective daily ingestion amount can be ingested. When the composition and agent according to the present invention are each provided in the form of a package, it is desirable that the package should give a statement about the ingestion amount so that the effective daily ingestion amount can be ingested, or that they should be provided together with a document which states the ingestion amount. When the composition and agent according to the present invention are each provided in a multiple package, a plurality of packages containing the effective daily ingestion amount may be provided as a set.

The predetermined ingestion amount per meal in the package form may be either the effective daily ingestion amount or an ingestion amount obtained by dividing the effective daily ingestion amount into two or more (preferably two or three) portions. Thus, the package form of the composition and agent according to the present invention can contain the medium chain fatty acid glyceride of the present invention in the daily ingestion amount for a human described above, or can contain the medium chain fatty acid glyceride of the present invention in an amount half or one third of the daily ingestion amount for a human described above. For convenience of ingestion, the composition and agent according to the present invention are each preferably provided in the form of a package in which the ingestion amount per meal is the effective daily ingestion amount.

The package form for providing the composition and agent according to the present invention is not particularly limited as long as it is a form that defines a constant amount, and examples of the package form include containers in which they can be contained, such as wrapping papers, bags, soft bags, paper containers, cans, bottles and capsules.

The composition and agent according to the present invention are each desirably administered or fed continuously for at least 1 week in order to more satisfactorily exert the effects thereof, and the administration or ingestion period is preferably continuously 4 weeks or longer (for example, 4 to 12 weeks), more preferably continuously 5 weeks or longer (for example, 5 to 12 weeks). The “continuously,” as used herein, means that administration or ingestion is continued at least once (once to seven times) weekly. When the composition and agent according to the present invention are each provided in the package form, packages containing an effective ingestion amount for a certain period (for example, 1 week) may be provided as a set, for continuous ingestion.

The food of the present invention may be attached with an indication that it has any or all of the effects for suppressing fat accumulation, suppressing body weight gain and promoting the production of a ketone body. In this case, the food of the present invention may be attached with part or all of the following indications for better understanding of consumers:
for persons who are anxious about body fat;
for persons who are anxious about visceral fat;
for persons who are anxious about body weight;
for persons who are plump;
for persons who are anxious about fatness;
helping to reduce abdominal fat;
helping to reduce waist circumference; and
useful for improving BMI value.

According to another aspect of the present invention, there are provided a method for suppressing fat accumulation, a method for suppressing body weight gain and a method for promoting the production of a ketone body, each comprising feeding or administering an effective amount of the medium chain fatty acid glyceride of the present invention or a composition comprising the medium chain fatty acid glyceride to a subject in need thereof. Also, according to the present invention, there are provided a method for treating, preventing or improving obesity and a method for reducing a risk of developing obesity, each comprising feeding or administering an effective amount of the medium chain fatty acid glyceride of the present invention or a composition comprising the medium chain fatty acid glyceride to a subject in need thereof. The method of the present invention can be carried out according to the descriptions about the composition and agent according to the present invention.

According to still another aspect of the present invention, there is provided use of the medium chain fatty acid glyceride of the present invention, for the manufacture of an agent for suppressing fat accumulation, for the manufacture of an agent for suppressing body weight gain, or for the manufacture of an agent for promoting the production of a ketone body. According to still another aspect of the present invention, there is provided use of the medium chain fatty acid glyceride of the present invention, as an agent for suppressing fat accumulation, as an agent for suppressing body weight gain, or as an agent for promoting the production of a ketone body. The use of the present invention can be carried out according to the descriptions about the composition and agent according to the present invention.

According to still another aspect of the present invention, there is provided use of the medium chain fatty acid glyceride of the present invention, for the manufacture of an agent for treating, preventing or improving obesity, or for the manufacture of an agent for reducing a risk of developing obesity. According to still another aspect of the present invention, there is provided use of the medium chain fatty acid glyceride of the present invention, as an agent for treating, preventing or improving obesity, or as an agent for reducing a risk of developing obesity. The use of the present invention can be carried out according to the descriptions about the composition and agent according to the present invention.

According to still another aspect of the present invention, there is provided a medium chain fatty acid glyceride comprising a saturated fatty acid having 8 carbon atoms as a constituent fatty acid, for use in suppressing fat accumulation, for use in suppressing body weight gain, or for use in promoting the production of a ketone body. According to still another aspect of the present invention, there is provided a medium chain fatty acid glyceride comprising a saturated fatty acid having 8 carbon atoms as a constituent fatty acid, for use in treating, preventing or improving obesity, or for use in reducing a risk of developing obesity. The medium chain fatty acid glyceride of the present invention can be carried out according to the descriptions about the composition and agent according to the present invention.

The method of the present invention and the use of the present invention may be use in mammals including humans and are intended to involve both of therapeutic use and non-therapeutic use. The “non-therapeutic,” as used herein, means elimination of operating, treating or diagnosing activities to a human (i.e., medical activities to a human), and specifically means elimination of a method of performing operation or treatment of, or diagnosis involving, a human by a doctor or a person who receives an instruction from the doctor.

### EXAMPLES

Hereinafter, the present invention will be described in more detail by way of the following examples, but is not limited thereto.

### Example 1: Effect of MCT on Suppressing Body Weight Gain and Suppressing Fat Accumulation

### (1) Test Method

Seven (7)-week-old C57BL/6J male mice (Japan SLC, Inc.) were acclimated with CLEA Rodent Diet CE-2 (manufactured by CLEA Japan, Inc.) for 1 week, and divided into 5 groups (n=9 or 10). Any of feeds having the compositions indicated in Tables 1 and 2, i.e., four types of test diets and a normal diet, was given to the respective groups for 5 weeks. Specifically, as the test diets, MCT diets were prepared by blending MCTs and LCT in a low-carbohydrate high-fat diet feed made based on an AIN-93G standard composition feed, and an LCT diet was prepared by blending LCT (manufactured by Wako Pure Chemical Industries) alone, without MCT, in a low-carbohydrate high-fat diet feed made based on an AIN-93G standard composition feed.

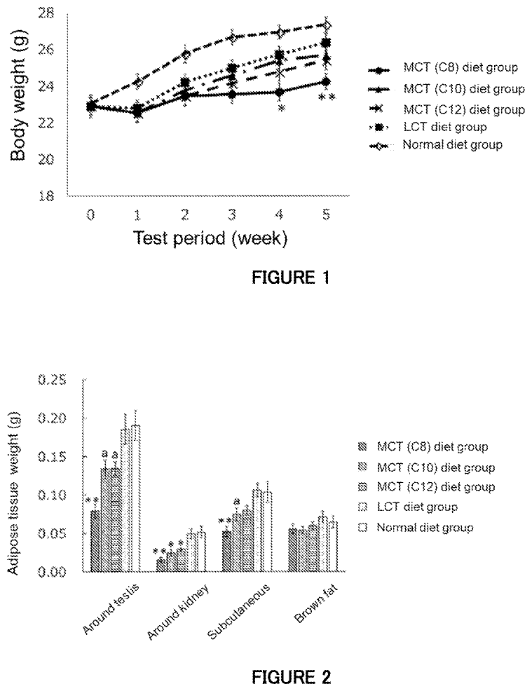

Since the ingestion amount of phosphorus (P), 860.4 mg, derived from casein reduced along with the reduction in amount of casein blended in the test diets as compared with the normal diet as shown in FIG. 1, potassium phosphate (KH₂PO₄, phosphorus ingestion source in AIN-93G) was blended in the respective test diets so that the phosphorus ingestion amounts from the normal diet and the test diets were equal to each other. To prevent the potassium (K) ingestion amounts from the test diets from increasing due to the blending of potassium phosphate (KH₂PO₄) described above as compared with the normal diet, the content of tripotassium citrate monohydrate (K₃C₆H₅O₇.H₂O) in a modified AIN-93G mineral mix blended in the test diets was reduced as shown in Table 2 so that the potassium ingestion amounts from the normal diet and the test diets were equal to each other. It should be noted that sucrose was blended in the same amount as the decrement of the content of tripotassium citrate monohydrate (K₃C₆H₅O₇.H₂O) in the modified AIN-93G mineral mix to make the total amounts equal to each other (Table 2).

The following three types of MCT diets were provided: “MCT (C8) diet” blended with a triglyceride comprising a fatty acid having 8 carbon atoms, caprylic acid, as a constituent fatty acid (manufactured by Tokyo Chemical Industry Co., Ltd.); “MCT (C10) diet” blended with a triglyceride comprising a fatty acid having 10 carbon atoms, capric acid, as a constituent fatty acid (manufactured by Tokyo Chemical Industry Co., Ltd.); and “MCT (C12) diet” blended with a triglyceride comprising a fatty acid having 12 carbon atoms, lauric acid, as a constituent fatty acid (manufactured by Tokyo Chemical Industry Co., Ltd.). The AIN-93G standard composition feed (manufactured by Meiji Co., Ltd.) was used as the normal diet. The fat component of the three types of MCT diets, the LCT diet and the normal diet was LCT derived from soybean oil. During the test period, the mice were allowed to freely eat the feed and drink water.

TABLE 1
Table 1: Compositions of normal diet and test diets

| | Normal | Test diet | | | |
|---|---|---|---|---|---|
| | diet | | MCT | MCT | MCT |
| | (AIN- | LCT | (C8) | (C10) | (C12) |
| | 93G) | diet | diet | diet | diet |
| Casein (g) | 200 | 80.5 | 80.5 | 80.5 | 80.5 |
| L-cystine (g) | 3 | 1.2 | 1.2 | 1.2 | 1.2 |
| Corn starch (g) | 400 | 0 | 0 | 0 | 0 |
| α-Corn starch (g) | 132 | 0 | 0 | 0 | 0 |
| Sucrose (g) | 98.95 | 29.45 | 29.45 | 29.45 | 29.45 |
| Soybean oil (g) | 70 | 391.2 | 174.9 | 174.9 | 174.9 |
| MCT* (g) | 0 | 0 | 216.3 | 216.3 | 216.3 |
| Cellulose (g) | 50 | 50 | 50 | 50 | 50 |
| AIN-93G mineral | 35 | 0 | 0 | 0 | 0 |
| mix (g) | | | | | |
| Modified AIN-93G | 0 | 35 | 35 | 35 | 35 |
| mineral mix (g) | | | | | |
| AIN-93G vitamin | 10 | 10 | 10 | 10 | 10 |
| mix (g) | | | | | |
| Potassium phosphate | 0 | 3614.1 | 3614.1 | 3614.1 | 3614.1 |
| (KH₂PO₄) (mg) | | | | | |
| Total (g) | 999.0 | 601.0 | 601.0 | 601.0 | 601.0 |
| Total energy (kcal) | 4005 | 4040 | 4040 | 4040 | 4040 |
| Protein (% kcal) | 20 | 8 | 8 | 8 | 8 |
| Lipid (% kcal) | 16 | 87 | 87 | 87 | 87 |
| Carbohydrate | 64 | 5 | 5 | 5 | 5 |
| (% kcal) | | | | | |
| MCT caloric ratio | 0 | 0 | 48 | 48 | 48 |
| LCT caloric ratio | 16 | 87 | 39 | 39 | 39 |
| | | | *MCT is | *MCT is | *MCT is |
| | | | tricaprylic | tricapric | trilauric |
| | | | acid. | acid. | acid. |

TABLE 2
Table 2: Compositions of AIN-93 mineral mix and its modified product

| | AIN-93G | Modified AIN-93G |
|---|---|---|
| Composition (mg/100 g) | mineral mix | mineral mix |
| CaCO₃ | 35,700 | 35,700 |
| KH₂PO4 | 19,600 | 19,600 |
| K₃C₆H₅O₇•H₂O | 7,078.0 | 4,073.7 |
| NaCl | 7,400 | 7,400 |
| K₂SO4 | 4,660 | 4,660 |
| MgO | 2,400 | 2,400 |
| FeC₆H₅O₇•XH₂O | 606 | 606 |
| ZnCO₃ | 165 | 165 |
| MnCO₃ | 63 | 63 |
| CuCO₃•CU(OH)₂•H₂O | 32.4 | 32.4 |
| KlO₃ | 1 | 1 |
| Na₂SeO₃ | 1.025 | 1.025 |
| (NH₄)₆Mo₇O₂₄•4H₂O | 0.795 | 0.795 |
| Na₂SiO₃•9H₂O | 145 | 145 |
| CrK(SO4)₂•12H₂O | 27.5 | 27.5 |
| LiC [symbol] | 1.74 | 1.74 |
| H₃BO₃ | 8.15 | 8.15 |
| NaF | 6.35 | 6.35 |
| NiCO₃•2Ni(OH)₂•4H₂O | 3.06 | 3.06 |
| NH₄VO₃ | 0.66 | 0.66 |
| Sucrose | 22,100.32 | 25,104.62 |
| Total | 100,000 | 100,000 |

[symbol]

indicates data missing or illegible when filed

The ingestion amounts of the test diets and the body weight changes during the test period (5 weeks) were measured, and the weights of the various organs including adipose tissues and the plasma levels of ketone bodies were measured at the end of the test period. Specifically, at the end of the test diet ingestion period, blood was collected from the abdominal aorta under Somnopentyl anesthesia by intraperitoneal administration, and adipose tissues around the testis and the kidney, subcutaneous adipose tissue, brown adipose tissue, and various organs were excised. In addition, plasma was collected from the collected blood, and ketone bodies were quantified according to an enzymatic method.

### (2) Results

The results were as shown in FIGS. 1 to 3. Among the test groups, no significant difference could be found in test diet ingestion amount or energy ingestion amount during the test period (data not shown).

FIG. 1 shows body weight transitions during the test period. The body weights of the test diet groups (4 groups) were changed to be low from 1 week after the beginning of ingestion as compared with the normal diet group. Especially, the MCT diet groups (3 groups) each showed a significantly low value as compared with the normal diet group, and the tendency was continued until 5 weeks after the beginning of ingestion. Among the test diet groups, the body weight gain of the MCT (C8) diet group was most prominently suppressed, and the body weights 4 weeks after and 5 weeks after the beginning of ingestion were significantly low as compared with the LCT diet group. Furthermore, among the MCT diet groups, the MCT (C8) diet group showed a tendency that the body weight gain was suppressed most. The MCT (C8) diet group did not exhibit any significant body weight gain during the test period as compared with the body weight at the beginning of the test (Week 0) (Dunnett's test). It should be noted that the MCT (C10) diet group exhibited a significant body weight gain (*p<0.05, Dunnett's test) from 3 weeks after the beginning of the test as compared with the body weight at the beginning of the test (Week 0), and that the MCT (C12) diet group exhibited a significant body weight gain (*p<0.05, Dunnett's test) from 4 weeks after the beginning of the test as compared with the body weight at the beginning of the test (Week 0). From the above results, it was confirmed that the suppressive effect on body weight gain differs depending on the number of carbon atoms of the constituent fatty acid of MCT, that is, caprylic acid triglyceride (C8) has a higher effect for suppressing body weight gain than those of capric acid triglyceride (C10) and lauric acid triglyceride (C12).

FIG. 2 shows body weights of various adipose tissues 5 weeks after the beginning of ingestion. No significant inter-group difference could be found in weights of various organs (pancreas, testis, kidney, colon, small intestine, cecum, liver, spleen and brain) other than fat 5 weeks after the beginning of ingestion (data omitted). On the other hand, the weights of fat around the testis, fat around the kidney and subcutaneous fat of the MCT diet groups (3 groups) were significantly low as compared with the normal diet group and the LCT diet group. Further, among the MCT diet groups, the MCT (C8) diet group showed a more prominent reduction in white adipose tissue weight as compared with the MCT (C10) diet group and the MCT (C12) diet group. When the normal diet group and the test diet groups (4 groups) were compared in brown adipose tissue (which burns fat to produce heat) weight, no difference could be found among the test diet groups. So, their heat producing functions were confirmed to be equivalent to each other. From the above results, it was confirmed that the suppressive effect on fat accumulation differs depending on the number of carbon atoms of the constituent fatty acid of MCT, that is, caprylic acid triglyceride (C8) has a higher effect for suppressing neutral fat accumulation than that of capric acid triglyceride (C10) or lauric acid triglyceride (C12). From these results and the results of the body weight transitions during the test period, it was suggested that caprylic acid triglyceride (C8) has a higher effect for suppressing neutral fat accumulation than that of capric acid triglyceride (C10) or lauric acid triglyceride (C12), and thus significantly suppresses body weight gain.

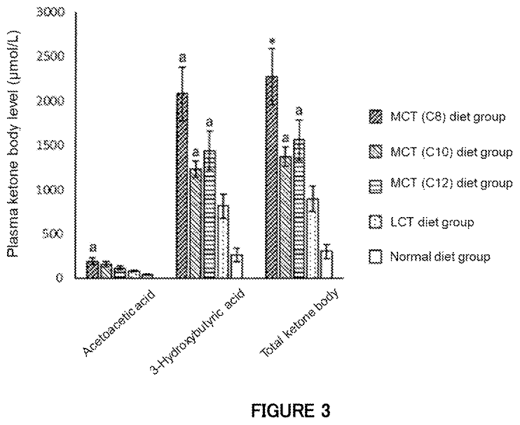

FIG. 3 shows plasma levels of ketone bodies 5 weeks after the beginning of ingestion. Both the plasma levels of ketone bodies, i.e., acetoacetic acid and 3-hydroxybutyric acid, of the test diet groups (4 groups) were significantly high, as compared with the normal diet group. Furthermore, among the test diet groups, the ketone body levels of the MCT (C8) group were highest, and significantly increased as compared with the LCT diet group. From the above results, it was suggested that caprylic acid triglyceride (C8) produces ketone bodies more than those produced by capric acid triglyceride (C10) or lauric acid triglyceride (C12), and thus significantly suppresses body weight gain and suppresses fat accumulation more.

1-17. (canceled)

18. A method for suppressing fat accumulation, a method for suppressing body weight gain and a method for promoting the production of a ketone body, each comprising feeding or administering an effective amount of a medium chain fatty acid glyceride comprising a saturated fatty acid having 8 carbon atoms as a constituent fatty acid or a composition comprising the medium chain fatty acid glyceride to a subject in need thereof.

19. A method for treating, preventing or improving obesity and a method for reducing a risk of developing obesity, each comprising feeding or administering an effective amount of a medium chain fatty acid glyceride comprising a saturated fatty acid having 8 carbon atoms as a constituent fatty acid or a composition comprising the medium chain fatty acid glyceride to a subject in need thereof.

20-23. (canceled)

24. The method according to claim 18, wherein the ratio (by mass, in terms of solid content) of the amount of the saturated fatty acid having 8 carbon atoms to the total amount of medium chain fatty acids of the medium chain fatty acid glyceride is 0.6 or more.

25. The method according to claim 18, wherein the medium chain fatty acid glyceride further comprises, as a constituent fatty acid, a saturated fatty acid having 10 carbon atoms and/or a saturated fatty acid having 12 carbon atoms.

26. The method according to claim 18, wherein the medium chain fatty acid glyceride or the composition is provided to a subject so that the medium chain fatty acid glyceride is ingested in a daily amount of 3 g or more (in terms of solid content).

27. The method according to claim 18, wherein the medium chain fatty acid glyceride or the composition is provided to a subject so that the medium chain fatty acid glyceride is ingested in an amount equivalent to 1 to 95% kcal of the total lipid energy ingestion amount of the subject.

28. The method according to claim 18, wherein the composition further comprises a long chain fatty acid glyceride.

29. The method according to claim 28, wherein the ratio (by mass, in terms of solid content) of the medium chain fatty acid glyceride to the long chain fatty acid glyceride in the composition is 1:0.1 to 1.5.

30. The method according to claim 18, wherein the composition further comprises a protein and/or a saccharide.

31. The method according to claim 29, wherein the composition further comprises a protein and/or a saccharide, and the ratio (by mass, in terms of solid content) of the total mass of the medium chain fatty acid glyceride and the long chain fatty acid glyceride to the total mass of the protein and the saccharide is 0.1 or more.

32. The method according to claim 29, wherein the composition further comprises a protein and/or a saccharide, and the contents of the medium chain fatty acid glyceride, the long chain fatty acid glyceride, the protein and the saccharide in the composition are 30 to 90% kcal, 5 to 50% kcal, 0 to 15% kcal and 0 to 10% kcal, respectively, with respect to the energy of the entire composition.

33. The method according to claim 18, wherein the medium chain fatty acid glyceride is a medium chain fatty acid triglyceride.

34. The method according to claim 18, wherein the long chain fatty acid glyceride is a long chain fatty acid triglyceride.

35. The method according to claim 18, wherein the composition is a food composition, a nutritional composition, or an oil and/or fat substitute.

36. The method according to claim 29, wherein the medium chain fatty acid glyceride is a medium chain fatty acid triglyceride, and the long chain fatty acid glyceride is a long chain fatty acid triglyceride.

37. The method according to claim 19, wherein the composition further comprises a long chain fatty acid glyceride.

38. The method according to claim 37, wherein the ratio (by mass, in terms of solid content) of the medium chain fatty acid glyceride to the long chain fatty acid glyceride in the composition is 1:0.1 to 1.5.

39. The method according to claim 38, wherein the medium chain fatty acid glyceride is a medium chain fatty acid triglyceride, and the long chain fatty acid glyceride is a long chain fatty acid triglyceride.
